(19) United States
(12) Patent Application Publication
(10) Pub. No.: US 2021/0315658 A1
(43) Pub. Date: Oct. 14, 2021

(54) AEROSOL SHIELD AND METHOD OF ASSEMBLY

(71) Applicants: David Censi, Brooklyn, NY (US); Barry Censi, Brooklyn, NY (US)

(72) Inventors: David Censi, Brooklyn, NY (US); Barry Censi, Brooklyn, NY (US)

(21) Appl. No.: 16/848,167

(22) Filed: Apr. 14, 2020

(51) Int. Cl. A61B 90/00

(52) U.S. Cl. CPC A61B 90/05

(57) ABSTRACT

A foldable shield for protecting physicians is disclosed. The foldable shield includes a first panel, a second panel, a third panel, and a fourth panel. The second panel is disposed adjacent the first panel and is foldable relative to the first panel along a first fold line between a first position and a second position. The third panel is disposed adjacent the first panel and is foldable relative to the first panel along a second fold line between a first position and a second position. The fourth panel is disposed adjacent the first panel and is foldable relative to the first panel along a third fold line between a first position and a second position. The fourth panel defines a first opening and a second opening therethrough.

### BACKGROUND

The present disclosure relates to shields for protecting people from a patient, and more particularly, to shields for protecting physicians, nurses, etc. from aerosol spray from a patient, and methods of assembling the shields.

Various types of personal protective equipment (“PPE”) are often worn by doctors, nurses, surgeons, staff, etc. (collectively referred to herein as “physicians”) to help provide protection from patients. In instances where the physician is not wearing sufficient PPE and/or when the patient has a particularly transferrable, contagious or harmful virus, for example, an additional shield between the physician and the patient may be helpful. Further, certain procedures performed by the physician may subject that physician to relatively large amounts of aerosol emitted from the patient. They physician may also benefit from an additional shield during these procedures.

Additionally, since the shield would likely be contaminated by the patient, it would be beneficial for the shield to be disposable. Accordingly, it is envisioned that a hospital caring for many patients would require many such shields. Thus, it would be helpful if the shields were relatively low-cost, easy to ship, easy to store. Even further, as time is typically of the essence in hospital settings, the ability to quickly and easily assembly and set up the shield would also be beneficial.

### SUMMARY

The present disclosure relates to a foldable shield for protecting physicians. The foldable shield includes a first panel, a second panel, a third panel, and a fourth panel. The second panel is disposed adjacent the first panel and is foldable relative to the first panel along a first fold line between a first position and a second position. The third panel is disposed adjacent the first panel and is foldable relative to the first panel along a second fold line between a first position and a second position. The fourth panel is disposed adjacent the first panel and is foldable relative to the first panel along a third fold line between a first position and a second position. The fourth panel defines a first opening and a second opening therethrough.

In disclosed embodiments, the second panel is perpendicular to the first panel when the second panel is in its second position. It is also disclosed that the third panel is perpendicular to the first panel and parallel to the second panel when the second panel and the third panel are in their second positions. It is further disclosed that the fourth panel is perpendicular to the first panel, the second panel and the third panel when the second panel, the third panel, and the fourth panel are in their second positions.

In disclosed embodiments, the foldable shield is made from a material including amorphous polyethylene terephthalate or polyethylene terephthalate. It is also disclosed that material is b about 0.010 inches and about 0.030 inches (e.g., 0.020 inches) thick

In embodiments, the second panel includes a foldable flap configured to adhere to the fourth panel. It is further disclosed that the third panel includes a foldable flap configured to adhere to the fourth panel. It is also disclosed that the second panel includes a second foldable flap configured to engage a flap of the fourth panel, and that the third panel includes a second foldable flap configured to engage the flap of the fourth panel.

In disclosed embodiments, the foldable shield consists of four panels including the first panel, the second panel, the third panel, and the fourth panel.

The present disclosure also relates to a method of assembling a foldable shield. The method includes folding a second panel of the foldable shield along a first fold line relative to a first panel of the foldable shield between a first position and a second position, folding a third panel of the foldable shield along a second fold line relative to the first panel of the foldable shield between a first position and a second position, folding a fourth panel of the foldable shield along a third fold line relative to the first panel of the foldable shield between a first position and a second position, the fourth panel defining a first opening and a second opening therethrough, exposing a first adhesive on a portion of the second panel, exposing a second adhesive on a portion of the third panel, adhering the second panel to the fourth panel with the first adhesive, and adhering the third panel to the fourth panel with the second adhesive.

In disclosed embodiments, the method includes folding a flap of the second panel where the first adhesive is disposed on the flap of the second panel. In embodiments, the method includes folding a flap of the third panel, where the second adhesive is disposed on the flap of the third panel.

It is also disclosed that the second panel is perpendicular to the first panel when the second panel is in the second position. Additionally, it is disclosed that the third panel is perpendicular to the first panel when the third panel is in the second position. It is further disclosed that the fourth panel is perpendicular to the first panel when the fourth panel is in the second position, and that the second panel is parallel to the third panel when the second panel is in the second position and when the third panel is in the second position.

### BRIEF DESCRIPTION OF THE DRAWINGS

An embodiment of the present disclosure is described hereinbelow with reference to the drawings wherein:

FIGS. 1-7 are perspective views of a shield during various stages of assembly in accordance with the present disclosure; and

FIG. 8 is a schematic view of the assembled shield of FIG. 7 positioned on a hospital bed, around portions of a patient, and with a physician's hands extending through openings of the shield.

### DETAILED DESCRIPTION

An embodiment of the presently disclosed shield and method of assembly are now described in detail with reference to the drawings, in which like reference numerals designate identical or corresponding elements in each of the several views. As used herein the term “distal” refers to that portion of the shield, or component thereof, farther from a user (e.g., physician), while the term “proximal” refers to that portion of the shield, or component thereof, closer to the user.

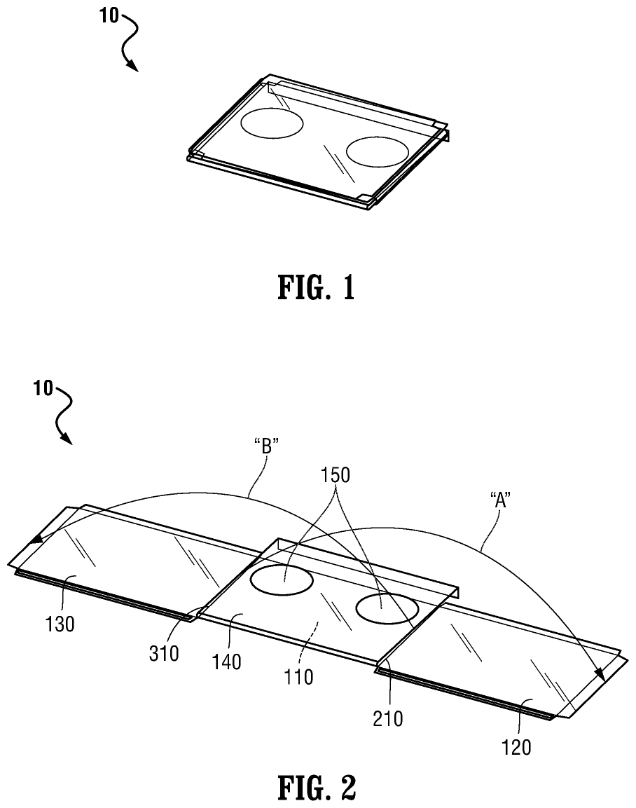

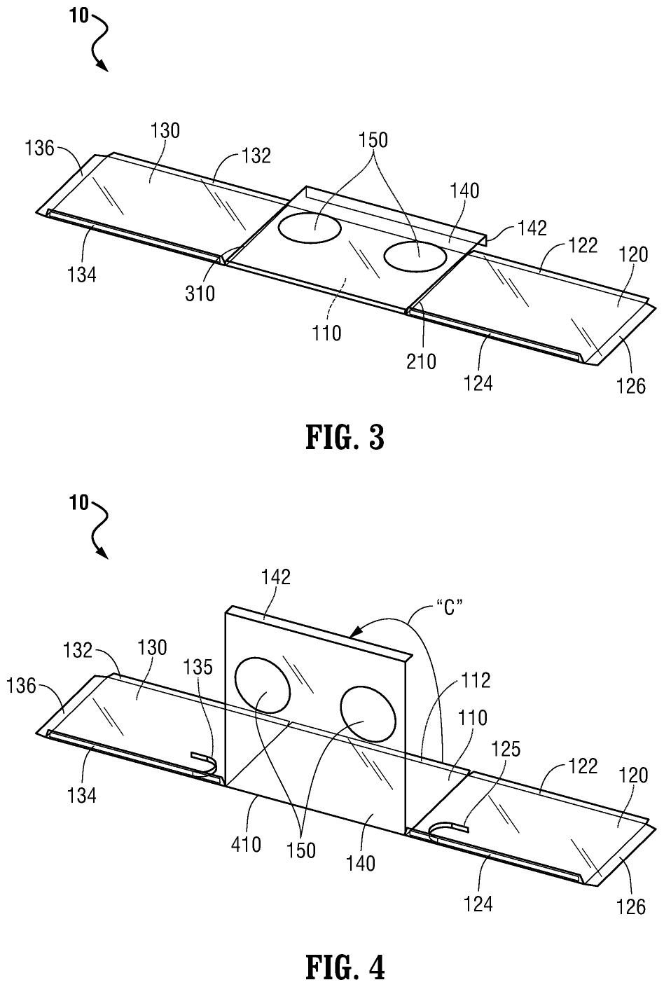

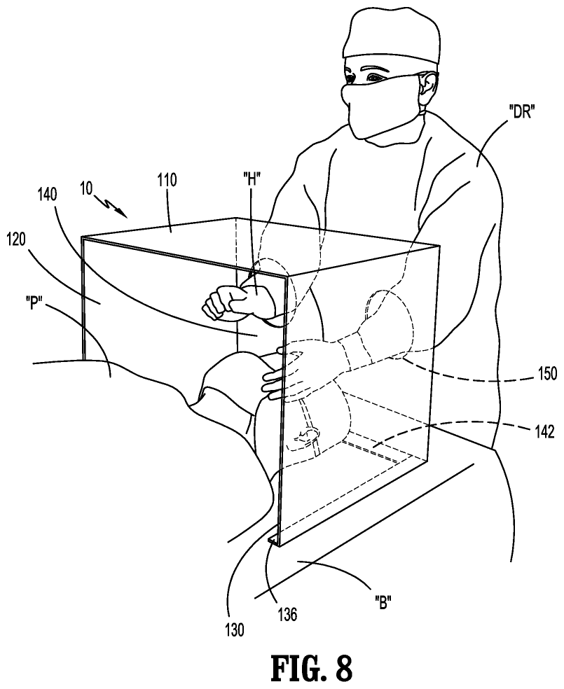

FIGS. 1-7 illustrate various stages of assembly of a shield for protecting physicians; the shield is generally referenced by numeral 10. As will be discussed in further detail below, the shield 10 is configured to be assembled rapidly and without the use of tools. In use, and as shown in FIG. 8, the shield 10 is configured to be positioned on a hospital bed “B” adjacent the head of a patient “P,” and is configured to allow a physician “DR” to place his or her hands “H” through openings 150 defined by the shield 10 to gain access to the patient “P.”

The shield 10 includes a plurality of panels which are foldable relative to each other, such that the shield 10 is assembled from a generally flat orientation (FIG. 1) to a box-like orientation (FIGS. 6-8). More particularly, the shield 10 includes a first panel 110, a second panel 120, a third panel 130, and a fourth panel 140. The second panel 120 is foldable relative to the first panel 110 along a first fold line 210. The third panel 130 is foldable relative to the first panel 110 along a second fold line 310. The fourth panel 140 is foldable relative to the first panel 110 along a third fold line 410. In embodiments, some or all of the fold lines 210, 310, 410 may be pre-folded, perforated, etc. to assist with the assembling of the shield 10.

The shield 10 also includes a plurality of flaps which are foldable relative to an associated panel such that they extend perpendicularly or generally perpendicularly from an associated panel. The plurality of flaps helps the panels remain in their desired orientation. More particularly, the first panel 110 includes a first flap 112; the second panel 120 includes a first flap 122, a second flap 124 and a third flap 126; the third panel 130 includes a first flap 132, a second flap 134 and a third flap 136; and the fourth panel 140 includes a first flap 142. All of the movable flaps are foldable or bendable relative to their associated panel in a direction that is generally toward the inside of the assembled shield 10. Most of the flaps are omitted in FIG. 8 for clarity.

The first flap 112 of the first panel 110 is disposed along an edge of the first panel 110 and is foldable relative to the first panel 110. Alternatively, the first panel 110 may not include any flaps. The first flap 122 of the second panel 120 and the second flap 124 of the second panel 120 are disposed opposite from each other on lateral edges of the second panel 120, and the third flap 126 of the second panel 120 is disposed on an edge of the second panel 120 that is opposite from the first panel 110. The first flap 132 of the third panel 130 and the second flap 134 of the third panel 130 are disposed opposite from each other on lateral edges of the third panel 130, and the third flap 136 of the third panel 130 is disposed on an edge of the third panel 130 that is opposite from the first panel 110. The first flap 142 on the fourth panel 140 is disposed along an edge of the fourth panel 140 that is opposite from the first panel 110.

It is envisioned that some, none or all of the flaps are pre-folded or pre-bent into shape, such that they are relatively stationary relative to their respective panel. For instance, the first flap 142 on the fourth panel 140 may be pre-folded relative to the fourth panel 140, as shown in the generally flat orientation of the shield 10 in FIG. 1.

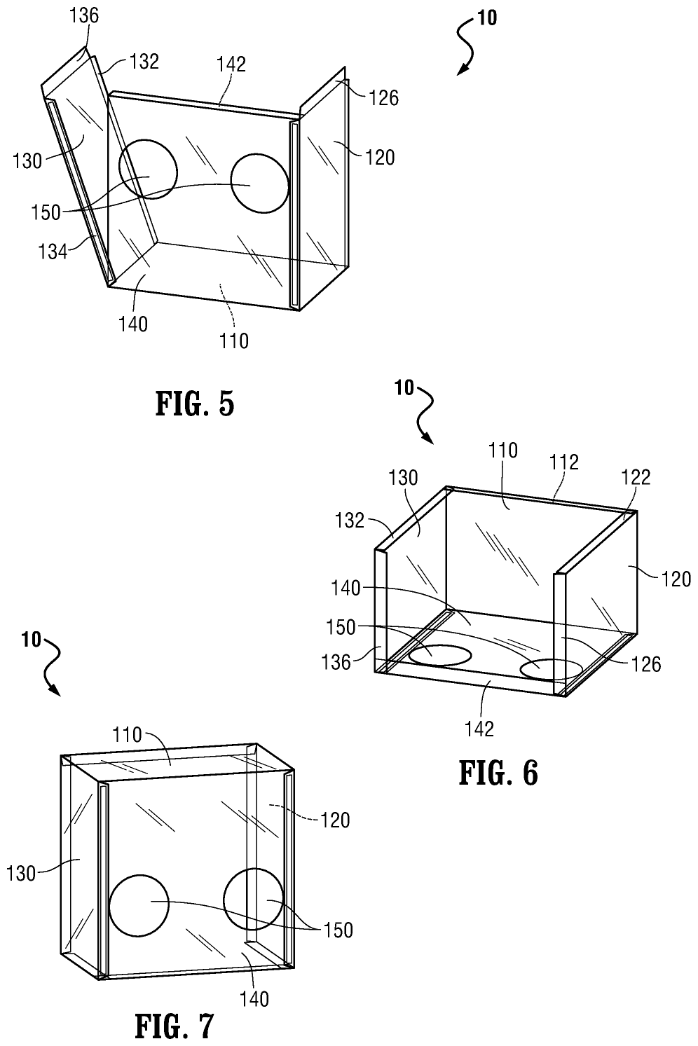

It is further envisioned that some, none or all of the flaps include adhesive thereon to enable the flap to adhere to an adjacent flap and/or to a surface (e.g., a hospital bed). Additionally, the adhesive may be covered by a removable peel. For instance, and with particular reference to FIG. 4, the second flap 124 of the second panel 120, and the second flap 134 of the third panel 130 each include adhesive thereon which is covered by respective peels 125, 135. As shown in FIG. 5, after the second panel 120 and the third panel 130 are folded relative to the first panel 110, the adhesive enables the second flap 124 of the second panel 120 and the second flap 134 of the third panel 134 to adhere to the first panel 110, thereby restricting movement therebetween.

The fourth panel 140 includes a pair of openings 150 extending therethrough. Each opening 150 is sized to allow a physician's hand, wrist and/or forearm to extend therethrough to gain access to the patient “P,” as shown in FIG. 8.

With continued reference to FIG. 8, the shield 10 is configured to allow a physician “DR” to access a patient “P” through the openings 150 in the fourth panel 140, while being protected from liquid aerosol spray emitted from the patient “P,” for example. The aerosol spray may be emitted from the patient “P” during breathing, speaking, coughing, sneezing, etc. Additionally, certain medical procedures, such as intubating a patient “P,” may cause fluid to be emitted or ejected from the patient's mouth or nose. The shield 10 helps protect the physician “DR” in each of these scenarios.

The shield 10 can be made from any suitable material, including materials that are disposable, inexpensive, relatively light weight, and transparent. For instance, in disclosed embodiments, the shield 10 is made from a plastic such as clear amorphous polyethylene terephthalate (APET), or polyethylene terephthalate (PET or PETE). The thickness of the material may be between about 0.010 inches and about 0.030 inches (e.g., about 0.020 inches), for example. As can be appreciated, other suitable materials and/or thicknesses may be used without departing from the scope of the present disclosure.

The present disclosure also includes a method of assembling the shield 10. With reference to FIG. 2, initially, the second panel 120 is folded relative to the first panel 110 in the general direction of arrow “A” such that the second panel 120 is co-planar with the first panel 110. Next, the third panel 130 is folded relative to the first panel 110 in the general direction of arrow “B” such that the third panel 130 is co-planar with the first panel 110 and the second panel 120. Next, the fourth panel 140 is folded relative to the first panel 110 in the general direction of arrow “C” in FIG. 4 such that the fourth panel 140 is perpendicular or generally perpendicular to the first panel 110. The peels 125, 135 are then removed from the second flap 124 of the second panel 120, and the second flap 134 of the third panel 130, respectively. Next, the second panel 120 is folded relative to the first panel 110 in the opposite direction of arrow “A” such that the second panel 120 is perpendicular or generally perpendicular to the first panel 110 and is perpendicular or generally perpendicular to the fourth panel 140. The third panel 130 is then folded relative to the first panel 110 in the opposite direction of arrow “B” such that the third panel 130 is perpendicular or generally perpendicular to the first panel 110 and is perpendicular or generally perpendicular to the fourth panel 140 (FIG. 5). All of the flaps can be folded relative to their respect panel at appropriate stages during assembly, such as in the order shown in FIGS. 1-7. After the peels 125, 135 are removed from their respective flaps 124, 134, the second flap 124 of the second panel 120 is adhered to the fourth panel 140 (e.g., to the outer-facing surface thereof), and the second flap 134 of the third panel 130 is adhered to the fourth panel 140 (e.g., to the outer-facing surface thereof). The assembly is then complete, and the shield 10 can be positioned on a hospital bed or another surface in the orientation shown in FIGS. 7 and 8.

Having an easy-to-assemble, easily shippable, and easily storable shield 10 would be beneficial to hospitals, surgery centers, etc. that would desire to have many shields 10 on site, such as one shield 10 per patient “P.” Further, such a shield 10 that is also relatively low cost would enable one shield 10 to be used for one patient “P” (as opposed to re-using shields), which would further protect physicians.

While the flaps are shown as being a particular size, it is envisioned and within the scope of the present disclosure that at least one flap is larger than illustrated, for instance. In such embodiments with larger flap(s), there would be additional overlap between some adjacent panels, thereby providing a more robust connection, for example.

It is also envisioned that the shield 10 includes covers for selectively covering the openings 150 of the fourth panel 140. The covers be foldable relative to the fourth panel 140 and/or the covers can utilize adhesive to adhere at least a portion of the cover to the fourth panel 140 to occlude the openings 150. It is envisioned that the covers can be used to occlude the openings 150 during transport of the patient “P,” or other times when the patent “P” is not being treated through the openings 150.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended hereto.

1. A foldable shield for protecting physicians, comprising:
a first panel;
a second panel disposed adjacent the first panel and being foldable relative to the first panel along a first fold line between a first position and a second position;
a third panel disposed adjacent the first panel and being foldable relative to the first panel along a second fold line between a first position and a second position; and
a fourth panel disposed adjacent the first panel and being foldable relative to the first panel along a third fold line between a first position and a second position, the fourth panel defining a first opening and a second opening therethrough.

2. The foldable shield according to claim 1, wherein the second panel is perpendicular to the first panel when the second panel is in its second position.

3. The foldable shield according to claim 2, wherein the third panel is perpendicular to the first panel and parallel to the second panel when the second panel and the third panel are in their second positions.

4. The foldable shield according to claim 3, wherein the fourth panel is perpendicular to the first panel, the second panel and the third panel when the second panel, the third panel, and the fourth panel are in their second positions.

5. The foldable shield according to claim 1, wherein the foldable shield is made from a material including amorphous polyethylene terephthalate or polyethylene terephthalate.

6. The foldable shield according to claim 5, wherein the material is between about 0.010 inches and about 0.030 inches thick. The foldable s according to claim 5, wherein the material is about 0.020 inches thick.

8. The foldable shield according to claim 1, wherein the second panel includes a foldable flap configured to adhere to the fourth panel.

9. The foldable shield according to claim 8, wherein the third panel includes a foldable flap configured to adhere to the fourth panel.

10. The foldable shield according to claim 9, wherein the second panel includes a second foldable flap configured to engage a flap of the fourth panel.

11. The foldable shield according to claim 10, wherein the third panel includes a second foldable flap configured to engage the flap of the fourth panel.

12. The foldable shield according to claim 1, wherein the foldable shield consists of four panels including the first panel, the second panel, the third panel, and the fourth panel.

13. A method of assembling a foldable shield, comprising:
folding a second panel of the foldable shield along a first fold line relative to a first panel of the foldable shield between a first position and a second position;
folding a third panel of the foldable shield along a second fold line relative to the first panel of the foldable shield between a first position and a second position;
folding a fourth panel of the foldable shield along a third fold line relative to the first panel of the foldable shield between a first position and a second position, the fourth panel defining a first opening and a second opening therethrough;
exposing a first adhesive on a portion of the second panel;
exposing a second adhesive on a portion of the third panel;
adhering the second panel to the fourth panel with the first adhesive; and
adhering the third panel to the fourth panel with the second adhesive.

14. The method according to claim 13, further including folding a flap of the second panel, wherein the first adhesive is disposed on the flap of the second panel.

15. The method according to claim 14, further including folding a flap of the third panel, wherein the second adhesive is disposed on the flap of the third panel.

16. The method according to claim 13, wherein the second panel is perpendicular to the first panel when the second panel is in the second position.

17. The method according to claim 16, wherein the third panel is perpendicular to the first panel when the third panel is in the second position.

18. The method according to claim 17, wherein the fourth panel is perpendicular to the first panel when the fourth panel is in the second position.

19. The method according to claim 18, wherein the second panel is parallel to the third panel when the second panel is in the second position and when the third panel is in the second position.
